(19) United States
(12) Patent Application Publication
(10) Pub. No.: US 2026/0123841 A1
(43) Pub. Date: May 7, 2026

(54) COMPUTER-IMPLEMENTED METHOD, COMPUTER PROGRAM PRODUCT, COMPUTER-READABLE MEDIUM, SYSTEM, AND METHOD FOR DETERMINING CARDIOVASCULAR PERFORMANCE

(71) Applicant: Nikolai MARINOW, Herrenberg (DE)

(72) Inventor: Nikolai MARINOW, Herrenberg (DE)

(21) Appl. No.: 19/349,357

(22) Filed: Oct. 3, 2025

(30) Foreign Application Priority Data
Nov. 5, 2024 (EP) 24210852.0

(51) Int. Cl. A61B 5/0205; A61B 5/00; A61B 5/021; A61B 5/024

(52) U.S. Cl. CPC A61B 5/0205; A61B 5/002; A61B 5/02141; A61B 5/02416; A61B 5/7267

(57) ABSTRACT

The invention relates to a computer-implemented method for determining a cardiovascular performance (2). The method comprises receiving a plurality of measurement data (5) through a communication interface (6) from a sensor (7) at a measurement section (41) of the patient's distal periphery (4). The measurement data (5) provides information about a perfusion of the blood flow at the measurement section (41). The plurality of measurement data (5) comprises at least two different measurement data (5) detected at different times after the blood flow to the distal periphery (4) has been restored. The method further comprises determining the cardiovascular performance (2), in particular the cardiac output, based on the plurality of measurement data and providing output data by an output interface (8) comprising the cardiovascular performance (2).

The invention relates to a computer-implemented method, a computer program product, a computer-readable medium, a system, and a method for determining a cardiovascular performance according to the independent claims. In addition, the invention relates to a computer-implemented method for training a machine learning model to determine a cardiovascular performance and a training data set according to the independent claims.

Cardiovascular performance, in particular stroke volume and/or cardiac output, is an important metric for assessing cardiac function, hemodynamic organ perfusion, and an indicator of cardiovascular health. In addition, cardiovascular performance monitoring can be used to check indications, such as the use of medication or therapeutic measures, for example to treat heart failure, shock or pulmonary hypertension. By monitoring cardiovascular performance, postoperative care can be monitored and/or the intended function of implants such as pacemakers can be checked.

US 2012/065514 A discloses a device comprising a pneumatic inflatable cuff and a Doppler ultrasound probe attachable to the wrist for measuring blood. This device allows the Doppler velocity of blood to be measured during deflation of the cuff.

However, a Doppler ultrasound probe is costly and difficult to position accurately and reliably, especially during physical exertion of a patient. In addition, US 2012/065514 does not disclose or teach a way to determine cardiovascular performance. Furthermore, reliable determination of cardiovascular performance in the prior art is often expensive, cumbersome, complex, time consuming and/or requires specialized personnel.

It is therefore the object of the present invention to overcome these and other disadvantages of the prior art and to provide computer-implemented methods, a computer program product, a computer-readable medium, a system, a method, and a training dataset which provide a simple, inexpensive, and reliable solution to the above problems. In addition, the determination of cardiovascular performance should be widely available and compatible with existing measurement methods/systems and preferably allow a simple and fast determination of cardiovascular performance during physical exertion. It is thus possible to determine cardiovascular performance and the associated diagnosis(es) even under heavy stress on the heart.

The computer-implemented method for determining a cardiovascular performance optionally comprises sending a first pressurization command, preferably by a control unit, to pressurize a cuff via a systolic blood pressure of the patient to prevent blood flow to a distal periphery of the patient. The computer-implemented method optionally comprises sending a second pressurization command, preferably by the control unit, to pressurize the cuff to a sub-systolic blood pressure to allow blood flow to the distal periphery. The computer-implemented method includes receiving, through a communication interface, a plurality of measurement data from a sensor at a measurement section of the distal periphery of the patient. The plurality of measurement data provide information about a perfusion of blood flow at the measurement section. The plurality of measurement data comprises at least two different measurement data detected at different times after restoring blood flow to the distal periphery. The computer-implemented method further comprises determining the cardiovascular performance based on the plurality of measurement data and providing output data through an output interface comprising the cardiovascular performance.

The cardiovascular performance may be cardiac output or stroke volume.

The cuff is preferably a blood pressure cuff. The cuff may be arrangeable around a part of the patient's body, in particular an extremity of the patient, preferably on the patient's upper arm at heart level. The dimension and shape of the cuff can be adapted to the patient's body part in order to enclose the body part along a contact area. A uniform surface pressure can be applied to the contact area when the cuff is pressurized.

Such a cuff, in particular a blood pressure cuff, is often already available in a clinical context for blood pressure measurements and is also widely used, so that both patients and clinicians accept it on the market. In addition, such a cuff improves the application of the computer-implemented method by reducing costs and training effort and ensuring compatibility with existing clinical procedures.

This computer-implemented procedure makes it possible to determine cardiovascular performance by restoring blood flow perfusion, which stabilizes in a saturation range only after a considerable time after blood flow restoration.

Optional control of the cuff for pressurization also allows for better tuning of the procedure and easier user application.

A patient's systolic blood pressure is typically around 120 mmHg, but can also be in the range of 90-180 mmHg depending on the patient. A first pressurization command to pressurize a cuff via systolic blood pressure, or arterial occlusion pressure, is therefore above these values so that the cuff can reliably prevent blood flow in the arteries.

The second pressurization command to pressurize the cuff to a sub systolic blood pressure in order to allow blood flow to the distal periphery is thus below the above-mentioned values. The second pressurization command can completely release the blood flow, i.e. achieve only a low pressurization or a pressurization pressure of 0 mmHg or at least below 40 mmHg of the pressure cuff. Alternatively, the second pressurization command to pressurize the cuff to a sub systolic blood pressure can enable a venous return stop. A venous return stop typically takes place in a pressurization of the cuff in a range of 40-60 mmHg.

The method may further comprise receiving blood pressure readings from the cuff, in particular through the communication interface. Thus, a time interval of the measurement can be easily and quickly selected.

The control unit can be a mobile terminal device, in particular a smartphone or a tablet.

The temporally consecutive plurality of different measurement data may have been recorded at a predetermined time distance from each other after the blood flow has been restored.

The plurality of measurement data for determining the cardiovascular performance may be selected over a time interval from a range of 1 s to 60 s, in particular from a range of 1 s to 35 s, preferably from a range of 1 s to 25 s. Alternatively, the time interval can be selected so that a deviation of measurement data directly following one another in time is always greater than 5%, in particular 4%, preferably 3%. In a further alternative, the time interval can be selected such that a mean value of three, four or five measurement data measured immediately following one another is greater than a predefined fraction, in particular 75%, preferably 85%, of a measurement data measured in immediate succession.

Depending on a pulse rate, in particular measured by the sensor, a number of measurement data corresponding to this time interval, which corresponds to the number of pulses in this time interval, can be selected to determine the cardiovascular performance.

This allows for quick and easy determination of the patient's cardiovascular performance using means that are substantially widely available and readily accessible in medical circles.

Blood perfusion saturation may vary in speed and/or overshoot depending on the patient and physical exertion of the patient. In particular, saturation of blood perfusion may only occur after a transient response in a transient range of blood perfusion. The transient response depends on the cardiovascular performance as well as the pulse rate.

It is therefore desirable if the time interval is determined by a termination criterion, such as the deviation of consecutive measured values.

A distance between a point of interrupted blood flow and the measurement section can be at least 3 cm, in particular at least 10 cm, preferably at least 15 cm.

This enables a more accurate and reliable measurement of the measurement data and thus determination of the cardiovascular performance, as the influence of disturbances, side effects and feedback at the site of the obstructed blood flow can be reduced. In addition, a greater spatial distance allows improved measurement of propagation effects. In particular, blood perfusion can be determined by the acceleration of blood flow after the restoration of blood flow. This acceleration can be modeled by a recursive function, for example. For greater distances from the site of interrupted blood flow, this acceleration is better reflected in the measurement data.

The plurality of measurement data may comprise or consist of measurement data that indicate the oxygen saturation in the blood, in particular SpO₂ measurement data, volumetric measurement data that indicate the perfusion amplitude in the measurement section, in particular perfusion index measurement data, and/or plethymographic measurement data that indicate the detection of a pulsation. Optionally, the measurement data can also include pulse rates.

The pulse rate is the number of heartbeats per minute of a patient.

This measurement data can be measured with simple and inexpensive means and is available and easily accessible in clinical practice.

Measurement data comprising pulse rate(s) is beneficial as cardiovascular performance is dependent on the patient's current pulse rate. In addition, cardiac output can be easily determined from cardiac output and pulse rate. The method may include determining a cardiac output as an intermediate step in determining the cardiac output, which is multiplied by the pulse rate to determine the cardiac output.

The pulse rate can be determined by the sensor or another sensor, in particular another sensor integrated in the cuff, and made available to the control unit.

In addition, patient-specific data can be provided to the control unit, which is taken into account when determining the cardiovascular performance in order to determine it more reliably. The patient-specific data may relate, for example, to a distance of the cuff from the measurement section, a height of the patient, and/or a weight of the patient. The sensor and the cuff may also be configured to automatically determine the distance of the cuff to the measurement section.

In addition, two or more sensors located at different areas of the patient's distal periphery may be used to provide a plurality of measurement data during a measurement to determine the cardiovascular performance of the control unit.

In addition to receiving measurement data from the sensor at the distal periphery where blood flow has been occluded, the method may include receiving a plurality of measurement data from another sensor located at another distal periphery where blood flow has not been/is not being occluded.

A perfusion amplitude, and in particular a stroke volume variability metric, may be determined from the plurality of measurement data, in particular from the SpO₂measurement data, the perfusion index measurement data, and/or the plethysmography measurement data.

The determination of a perfusion amplitude for each measured value of the measurement data makes it possible to measure a change in perfusion.

The stroke volume variability metric can result directly from the plethysmography measurement data. The stroke volume variability metric can be determined, for example, by a deviation of the measured perfusion index from a maximum value of the perfusion index in the time interval or average value of the perfusion index in the saturation range.

The stroke volume variability metric could also be determined by a deviation of a pulse pressure from a maximum value or average value of the pulse pressure.

Preferably, the stroke volume variability metric can be determined individually for each sensor pulse.

Especially in the area of a steep increase in the values of the measurement data after allowing blood flow to the distal periphery, the influence of the heartbeat volume variability is particularly large, while the influence decreases sharply later in the transient/saturation range. It is therefore desirable to take the heartbeat volume variability into account, especially in the area of the steep rise, in order to determine the cardiovascular performance. The measurement data, such as perfusion amplitudes, rise sharply even in short periods of a few seconds, so that small deviations could otherwise lead to relatively high measurement inaccuracies.

This can be particularly problematic for high pulse rates, as the measurement frequency of the sensor is often lower than the pulse rate.

The stroke volume variability metric can be used to correct the measurement data, in particular the perfusion amplitude. Thus, it is possible to correct for a fluctuation in stroke volume by taking into account the stroke volume variability metric, which can be influenced by, among other things, respiratory rhythm, cardiac arrhythmias, pathological conditions, blood volume status of the patient, thoracic pressure, body position, and/or movement.

Another significant advantage of the method is that any hemodynamically relevant cardiac malformations or pathologies do not have a distorting influence on the measurement at the distal periphery. In contrast, measurement methods close to the heart for determining cardiovascular performance are often directly influenced by such cardiac malformations or pathologies. This allows the method according to the invention to determine more reliable values for cardiovascular performance despite congenital heart defects (e.g. septal defect) or pathological changes (e.g. mitral regurgitation) that lead to a reduction in stroke volume (e.g. due to shunt formation). The part of the blood that does not flow into the systemic circulation has no effect in the distal periphery, but distorts cardiovascular performance measurements close to the heart. The change in perfusion amplitude, on the other hand, depends only on the actual blood flow arriving in the distal periphery.

The perfusion amplitude can be normalized. For example, a measured value can be normalized with the maximum value of the measured value in the time interval, a mean value in the transient range/saturation range of the measurement data, or the last measured value of the time interval. For example, the measured perfusion index can be normalized by a maximum perfusion index in the time interval. The measurement of the perfusion amplitude is therefore a relative measured value.

The perfusion amplitude may be determined from a ratio of a pulsatile component, which provides information about a change in light absorption and thus blood perfusion, and a non-pulsatile component, which provides information about the non-pulsatile elements, such as skin, bone, continuously flowing arterial blood, and venous blood.

The computer-implemented method may include determining cardiovascular performance using at least one perfusion amplitude fit function. The at least one fit function may comprise or consist of a polynomial fit function, a logarithmic fit function, an exponential fit function, in particular with oscillatory damping, a logistic growth model fit function, in particular with oscillatory damping, a power law saturation fit function, a Hill fit function, a differential equation fit function, in particular of second order.

The computer-implemented method may comprise a determination of cardiovascular performance using an algorithm comprising a provision of a plurality of reference functions, preferably comprising measurement data measured previously in time, in particular measurement data of a similar or the same pulse rate measured previously in time. This algorithm further comprises a determination of a deviation of the plurality of reference functions with the plurality of received measurement data or of correlation values of the measurement data, and a selection of a reference function of the plurality of reference functions with the least deviation or a most similar correlation value to the received measurement data.

The cardiovascular performance can be determined with the help of a perfusion amplitude averaged over a time interval of the measurement, which is offset against the pulse rate, in particular multiplied completely or proportionally by the pulse rate.

The cardiovascular performance can be determined using a machine learning model, in particular a pre-trained machine learning model, based on the perfusion amplitudes.

This at least one fit function enables a reliable fit of usual perfusion amplitude values. In particular, the fit function can be selected in such a way that a reliable fit of the perfusion amplitude values that overshoot a saturation range is made possible before the perfusion amplitude values fluctuate only in the saturation range. In addition, the fit function can be selected in order to map that the perfusion amplitude reaches at least a local maximum in the transient range, then falls again and finally rises to the saturation range in order to fluctuate only in the saturation range. This can be done using higher order polynomials as a fit function.

The determination of cardiovascular performance using an averaged perfusion amplitude or a machine learning model enables a reliable and simple approximation of cardiovascular performance.

In this context, a similar pulse rate is a pulse rate that deviates in less than 5 beats per minute, preferably less than 3 beats per minute, more preferably less than 2 beats per minute, from the patient's pulse rate. Preferably, a similar pulse rate may deviate less than 1 beat per minute.

The correlation value may be about a correlation coefficient, in particular about the Spearman correlation coefficient.

The computer-implemented method may comprise selecting and/or combining one or more fit functions of the perfusion amplitude from a plurality of different fit functions.

As a metric for selecting the fit function or reference function, a deviation, in particular the mean absolute error or mean square error/root error, of the fit function or reference function with the perfusion amplitude of the measurement data can be determined. The metric can also be weighted, in particular weighted to give special consideration to the area of the strongest increase in perfusion amplitude and to give less consideration to saturation after restored blood flow. In particular, the deviation within the first 10 s, especially the first 8 s, preferably the first 4 s, of the time interval of the measurement can be weighted particularly heavily.

A sum/integration, over the perfusion amplitude, the reference function, and/or the fit function(s) until saturation of the perfusion amplitude is reached, can be used to determine the cardiovascular performance.

The measured values themselves only indicate a step function, as measured values are only available at discrete points, so that they are subject to a certain degree of inaccuracy. An integration of the fit function(s), on the other hand, can also take this transition between the individual measured values into account.

The fit function with the lowest deviation can be selected.

The slope, i.e. the first derivative, of the best fit function can be determined at certain points in time in order to determine the cardiovascular performance, in particular the cardiac output. The measurement of the slope may be performed at a time point representing an exceedance of a predetermined proportion to a saturation value.

The method may comprise using a plurality of fit functions independently to determine cardiac output.

The method may comprise averaging a sum of the normalized perfusion amplitude of the plurality of measurement data over a time interval.

The time interval of the metric can be determined using (i) a special algorithm, in particular a fit function, (ii) repeated measurements of measured values that do not deviate significantly from each other, or (iii) a time at which a predetermined proportion, in particular about 66%, of the maximum perfusion amplitude or a saturation value of the perfusion amplitude is exceeded.

For a more accurate measurement, the first measured value of the normalized perfusion amplitude can also be subtracted from the sum.

The averaged value of the perfusion amplitudes in the time interval of the measurement, or a predefined fraction thereof, can be multiplied by the pulse rate to approximately determine the stroke volume. The cardiac output can be approximately determined by multiplying it by the pulse rate again.

The distance between the first pressurization command and the second pressurization command can be at least 15 seconds, in particular at least 20 seconds, preferably at least 25 seconds. The time distance can be selected so that a measured value or a mean value of 2, 3, 4 or 5 measurement data, in particular perfusion index measurement data, measured in immediate succession is smaller than a predefined fraction, in particular 20%, preferably 15%, of a measured value or mean value of 2, 3, 4 or 5 measurement data measured in immediate succession that was measured before the pressurization.

Such an initial pressurization command can ensure that the perfusion of blood has been reliably stopped before the measurement begins shortly after blood flow has been restored. This enables an optimized measurement, especially for SpO₂measurement values.

Alternatively, the second pressurization command can be sent after the systolic blood pressure has been exceeded by the pressurizing cuff for a predefined period of time. The time period can be selected from 2 s-12 s, in particular 4 s-10 s, preferably 5 s-9 s. In particular, the time period can be 3 s, 4 s, 5 s, 6 s, 7 s, 8 s, 9 s, 10 s or 11 s.

The plurality of measurement data of the sensor can be detected at rest and/or during physical exertion of the patient.

Such a plurality of measurement data enables a more reliable determination of the cardiovascular performance(s), as the plurality of measurement data are detected in a controlled exercise. In addition, the detection of the plurality of measurement data with the previously described measurement data, in particular the SpO₂measurement data, is also easily possible during physical exertion, as these can be determined by compact and portable sensors.

Cardiovascular performance fluctuates considerably, for example an adult's cardiac output at rest can be around 4.5 L/min-5.5 L/min, while during physical exertion it increases to around 15 L/min-25 L/min. Thus, the measurement accuracy can be increased in particular by a large number of measurement data detected at rest and during physical exertion. The physical exertion can correspond to a predefined value, such as a standard ergometry test, in particular an ergometry test with a bicycle or a Bruce protocol ergometry test. In particular, the physical exertion may correspond to a value of 50 W-450 W, in particular 100 W-200 W, preferably 125 W-175 W.

The computer-implemented method may comprise a first determination of a first cardiovascular performance based on measurement data recorded at rest and a second determination of a second cardiovascular performance based on measurement data recorded during physical exertion of the patient.

Such a determination of the first and second cardiovascular performance allows for a more reliable assessment of the patient's cardiovascular health. In particular, possible conditions such as fever, hyperthyroidism, hypertension, heart failure, circulatory shock, aortic valve stenosis, or coronary artery disease, may directly or indirectly affect cardiovascular performance.

Another aspect of the invention is directed to a computer program product having instructions which, when the program is executed by a computer, cause the computer to perform the computer-implemented method described above.

Another aspect of the invention is directed to a computer-readable medium containing instructions which, when executed by a computer, cause the computer to perform the computer-implemented method described above.

A further aspect of the invention is directed to a system for carrying out the computer-implemented method described above, comprising means, in particular a communication interface, an output interface, and a control unit, for carrying out the computer-implemented method.

This allows to provide an integral solution of a system for performing the previously described method, to reduce the implementation effort, to optimize the compatibility and the functionality of the components, and to provide an improved user experience.

The system may include at least one sensor, connected or connectable to the control unit, for the distal periphery of a patient. The at least one sensor may be configured to measure a plurality of measurement data indicative of oxygen saturation in the blood. The at least one sensor may comprise or consist of a sensor for measuring volumetric measurement data, plethysmography measurement data, and optionally pulse rates.

Such sensors, in particular an SpO₂ sensor, are compact and easily integrated into technology, in particular wearable technology, and are cost effective. In particular, such sensors are more compact and easier to handle than, for example, a Doppler ultrasound meter, which could also be used for a blood perfusion measurement.

The method may comprise determining a slope metric using a plurality of measurement data measured chronologically in succession at different times. The slope metric is indicative of the slope of the plurality of measurement data measured chronologically in succession at different times. The method may comprise determining a characteristic value using the measurement data. The characteristic value is indicative of a saturation value or metric of the measurement data. The characteristic value may be indicative of an arithmetic metric, a quadratic metric, a median, or a variance. The method may comprise determining cardiovascular performance based on the slope metric and the characteristic value. In particular, a cardiovascular performance characteristic value may be determined using the slope metric and the characteristic value.

The cardiovascular performance may be provided by the cardiovascular performance parameter and at least one mapping logic.

This enables a simple, reproducible and reliable determination of the cardiovascular performance by determining only the slope metric and the characteristic value of the measurement data. This slope metric and the characteristic value allow measurement data, which is typically subject to multivariate influences, to be used particularly reliably to model cardiovascular performance, for example via the mapping logic.

Preferably, the slope metric is a metric of the mean slope of all measurement data in the time interval, as this reduces the impact of heart rate variability.

For example, the measurement data could be perfusion amplitudes. The characteristic value could be the arithmetic metric of the perfusion amplitude. The slope metric can be, for example, an average slope of the perfusion amplitude. This slope metric can preferably be determined for a number of measurement data/time interval of the measurement data immediately after the blood flow is restored. The number of measurement data/time interval of measurement data may be predefined or selected as previously described.

The cardiovascular performance is typically dependent on the pulse rate, but not necessarily linear. For example, the stroke volume may increase with increasing pulse rate but decrease for high pulse rates. The method may include using the pulse rate as a slope metric and characteristic value to provide an even more reliable determination of cardiovascular performance.

The cardiovascular performance can be determined using the measurement data and at least one mapping, in particular using at least one mapping logic. The at least one mapping can be determined using an ultrasound measurement method, a thermodilution method, a magnetic resonance imaging method, the Fick principle, a pulse contour analysis, VO₂max method, and/or bioimpedance methods for measuring cardiovascular performance.

The mapping logic can be a lookup function or table.

Cardiovascular performance in patients is typically determined using different measurement methods, which vary in reliability and are often subject to systematic fluctuations. Clinicians who use reference cardiovascular performance data from a particular measurement method for a patient therefore rely, for comparison, on the determination of the cardiovascular performance providing comparable values that are subject to similar systematic variations of the measurement method.

Determining cardiovascular performance from a mapping made using a specific measurement technique allows comparable values of cardiovascular performance to be provided.

In addition, magnetic resonance imaging has been found to provide particularly reliable and relatively easy to determine values of cardiovascular performance.

The method may comprise obtaining a user input indicative of the mapping to be used to determine cardiovascular performance, such as a mapping determined from at least one of the above measurement methods.

The method may comprise determining a plurality of cardiovascular performances determined using the measurement data and at least two different mappings. The output data may comprise a plurality of cardiovascular performances.

The at least one sensor may comprise or consist of a finger pulse oximeter, a wrist pulse oximeter, a wearable watch, in particular a smartwatch, or a fitness wristband with an integrated pulse oximeter sensor, a stationary pulse oximeter device with removable sensors, and/or a skin patch pulse oximeter.

Such sensors are particularly simple and/or practicable to use, user-friendly, and in particular have small dimensions.

The at least one sensor may be connected or connectable to the control unit by an electrical conductor or a wireless connection. In addition, the sensor may be connected to the cuff by an electrical conductor or a wireless connection.

The system may include a cuff. The cuff is pressurizable by a pressurization command from the control unit to stop and restore blood flow to a distal periphery of the patient.

This enables optimized interoperability of the cuff, the at least one sensor and the control unit and automated and/or synchronized performance of the procedure with reduced user effort.

The system may comprise a user interface, in particular comprising at least one or a touch screen and/or control elements, for user input. The system may be configured to perform the computer-implemented method described above fully or semi-automatically, in particular based on user input.

The cuff may be connected or connectable to the control unit by an electrical conductor or wireless connection.

The cuff may include a sensor for detecting measurement data comprising the pulse rate of the patient and providing this measurement data to the control unit.

The cuff may be configured to determine a systolic blood pressure of the patient upon pressurization and optionally provide this to a control unit.

Another aspect of the invention is directed at a method of determining cardiovascular performance from a plurality of measurement data. The method includes pressurizing a cuff over a systolic blood pressure of a patient to prevent blood flow to a distal periphery of the patient. The method includes pressurizing the cuff to a sub systolic blood pressure to allow blood flow to the distal periphery. The method includes measuring a plurality of measurement data from a sensor at a measurement section of the distal periphery of the patient. The measurement data provides information about a perfusion at the measurement section. The plurality of measurement data comprises at least two different measurement data detected at different times after restoring blood flow to the distal periphery. The method further comprises determining the cardiovascular performance, in particular the cardiac output, based on the plurality of measurement data.

Another aspect of the invention relates to a computer-implemented method for training a machine learning model for determining a cardiovascular performance, in particular the machine learning model described above. The computer-implemented method comprises receiving an input training dataset comprising measurement data and an associated target value of a cardiovascular performance. The measurement data was detected by at least one sensor at a measurement section of a distal periphery of a patient after restoring a blood flow of the distal periphery. The measurement data is preferably previously described measurement data. The computer-implemented method includes performing a training process of the machine learning model by predicting an output value, comparing the output value with the target value, adjusting the model parameters by a loss function. This training process is repeated iteratively for further input training datasets until a termination criterion is reached. Preferably, the trained model is validated by applying it to a separate validation dataset comprising validation measurement data and corresponding cardiovascular validation outputs to check the model. In addition, the trained model is provided.

Another aspect of the invention is directed to the training dataset/validation measurement dataset for use in the previously described computer-implemented method for training a machine learning model comprising at least one input training dataset.

The input training dataset may be indicative of blood oxygen saturation, in particular comprising measurement data indicative of blood oxygen saturation, preferably comprising or consisting of SpO₂ measurement data, volumetric measurement data, plethysmography measurement data, and preferably pulse rates.

The target value of the training data set can be determined by an ultrasound method, a thermodilution method, a magnetic resonance imaging method, the Fick principle, a pulse contour analysis, VO₂max method, and/or a bioimpedance measurement.

Preferably, the target value is determined by the magnetic resonance imaging method.

Further embodiments of the invention and improvements of the described embodiments will become apparent in the following description of the embodiments.

The invention will now be described with reference to certain embodiments and figures showing:

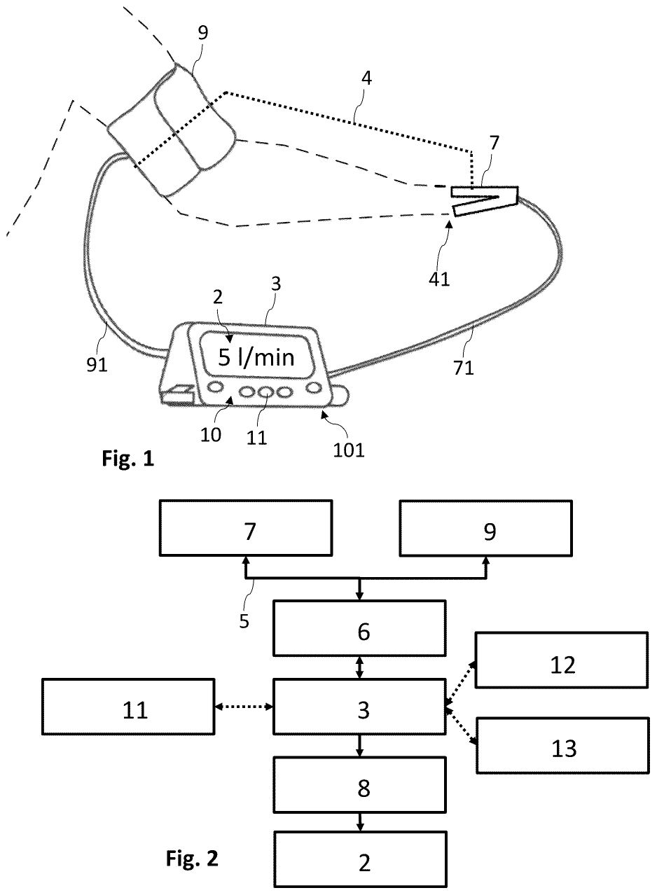

FIG. 1: A perspective view of a system according to the invention for determining cardiovascular performance according to a computer-implemented method according to the invention,

FIG. 2: a schematic representation of the computer-implemented method according to the invention,

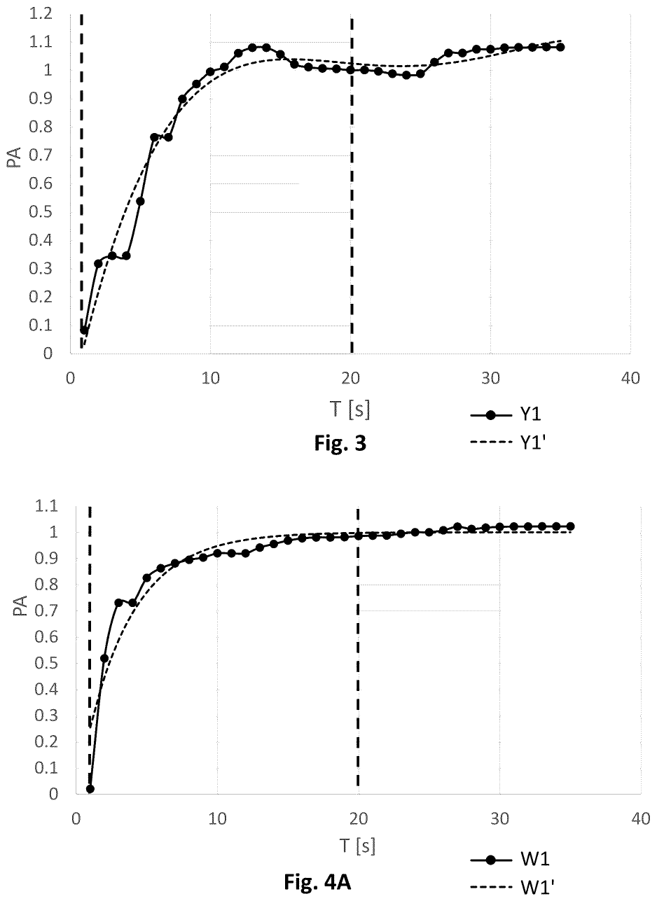

FIG. 3: a plurality of perfusion amplitudes of the measurement data over time, determined at rest of the patient and approximated by a polynomial fit function,

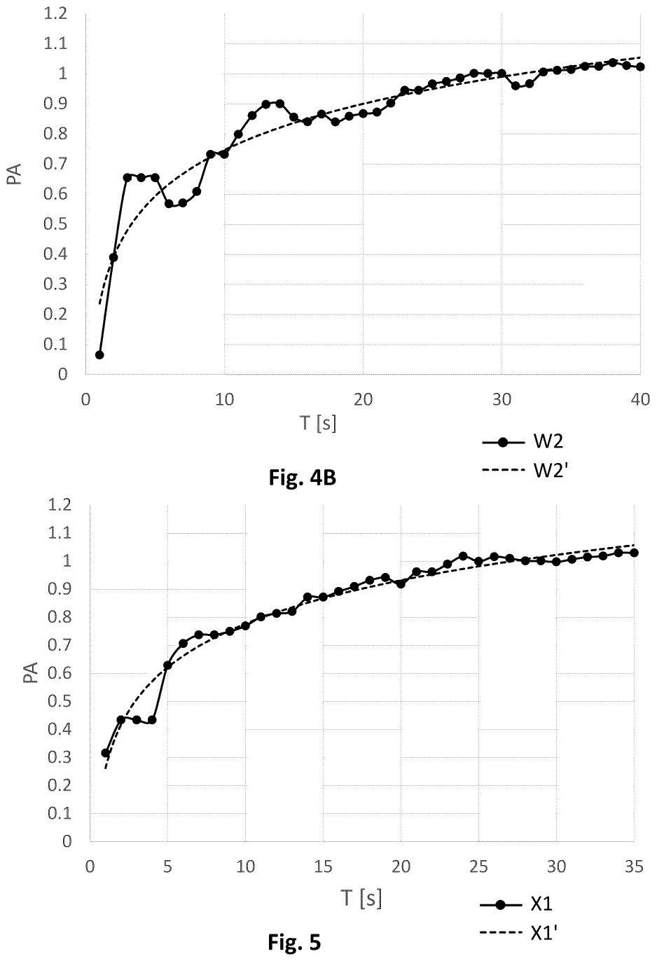

FIG. 4A: a plurality of perfusion amplitudes of the measurement data over time determined at rest of the patient and approximated by an exponential fit function,

FIG. 4B: a plurality of perfusion amplitudes of the measurement data over time with overshoots determined at rest of the patient and approximated by a logarithmic fit function,

FIG. 5: A plurality of perfusion amplitudes of the measurement data over time determined with the patient at physical exertion and approximated by a logarithmic fit function, and

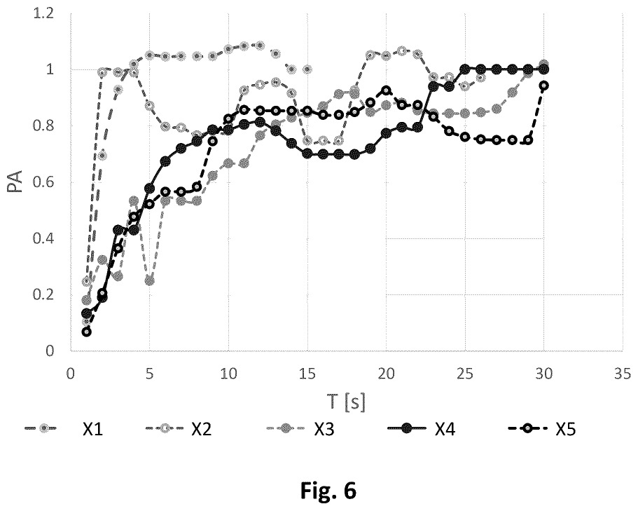

FIG. 6: a training data set over time, comprising exemplary five measurement series of perfusion amplitudes, for training the machine learning model.

FIG. 1 shows a perspective view of a system 101 according to the invention for determining cardiovascular performance in the form of cardiac output according to a computer-implemented method according to the invention. The system 101 comprises a control unit 3 connected via cables 71, 91 to a cuff 9 and an SpO₂ sensor 7. Alternatively, the cuff 9 and/or the SpO₂ sensor 7 may be wirelessly connected to the control unit 3. The SpO₂ sensor 7 is a finger pulse oximeter, which can be attached to a finger of a distal periphery 4 of a patient to determine the oxygen saturation in the blood at a measurement section 41. However, other sensors 7 can be used that are suitable for measuring blood perfusion.

The distal periphery 4 in the form of an upper limb of the patient and part of a contour of the patient are indicated by a dashed line in FIG. 1.

The cuff 9 may be attached to the patient's upper arm for restraining blood flow. The distance between the cuff 9 and the sensor 7 at the fingertip can thus be about 35 cm to 65 cm. Alternatively, a cuff 9 could be attached to the wrist and the sensor 7 to the fingertip. The distance between the cuff 9 and the sensor 7 can therefore be about 3 cm to 40 cm, in particular 10 cm to 30 cm, preferably 15 cm to 24 cm.

The control unit 3 is connected to a user interface 10, which comprises control elements 11 in the form of mechanical buttons. These operating elements 10 allow a user of the system 101 to start the measuring process.

Once the measurement process has been started, the measurement is preferably carried out fully automatically. The control unit 3 sends a first pressurization command to the cuff 9 via a communication interface 6 (see FIG. 2) so that the cuff 9 is pressurized via the systolic blood pressure of a patient so that the blood flow to the periphery 4 of the patient is prevented.

Subsequently, the control unit 3 sends a second pressurization command via a communication interface so that it is pressurized to a sub systolic blood pressure, enabling blood flow to the distal periphery 4, and, in particular, also enabling venous return.

The time distance between the first and second pressurization commands is selected such that a mean value of four perfusion index measured values immediately following one another is less than 20% of a perfusion index measured value immediately following these perfusion index measured values.

As an alternative to controlling the cuff 9 via the control unit 3, the cuff 9 can also be operated independently of the system 101, for example by a user manually or by another control unit. Once blood flow to the distal periphery 4 is released through the cuff 9, a plurality of perfusion index readings is detected by the SpO₂ sensor 7. For example, one time interval of the measurement is performed over 60 s. This time interval is selected so that a mean value of four measurement data immediately following one another is greater than 85% of the subsequent measured value.

A perfusion amplitude, i.e. a perfusion index normalized to a maximum value, is then determined for the measurement data. The perfusion amplitude can be normalized, for example by dividing the measured perfusion index values by a maximum value in the measurement interval, a final value in the measurement interval or an averaged saturation value of the measurement interval. The averaged saturation value can result from a metric of all measured values of a saturation range, in that the measured values only vary within a certain range.

The control unit 3 is configured to determine the cardiovascular performance 2 in the form of the cardiac output by means of these perfusion amplitude values using the methods described below (see FIG. 2).

Subsequently, the cardiac output, which may be 5 l/min, for example, is provided by an output interface 8 (see FIG. 2) and output on a display as shown in FIG. 1.

However, the computer-implemented procedure could also be carried out using a mobile device such as a smartphone as a control unit 3, for example with a software application for mobile devices. For this purpose, the cuff 9 and/or the sensor 7 could have a data transmission in the ISM frequency range, preferably in the range of 2.4 GHz, for communication with the mobile terminal device.

FIG. 2 shows a schematic representation of the computer-implemented method according to the invention. FIG. 2 shows a sensor 7 for recording SpO₂ measurement data 5, volumetric measurement data, and/or plethsymographic measurement data, and optionally a pulse rate, and a cuff 9 for pressurization to prevent blood flow to a distal periphery of a patient. The sensor 7 and the cuff 9 are uni- or bidirectionally connected to a communication interface 6. This measurement data 5 is converted into a perfusion amplitude by the control unit 3. The control unit 3 can both send pressurization commands to the cuff 9 via the communication interface 6 and preferably receive measurement data, in particular a pulse rate and/or a current pressurization value. This ensures, that the measurement only starts when pressurization of the cuff 9 with a target pressure has been achieved, i.e. a pressure above the patient's systolic blood pressure. In addition, the control unit 3 can receive a large amount of measurement data 5, which provides information about the perfusion of the blood flow at a measurement section, from the sensor 7 via the communication interface 6.

The control unit 3 can use a fit function 11, a machine learning model 12, a deterministic approximation method, and/or a reference function 13 to determine the cardiovascular performance 2 based on the measurement data 5 of a patient, as shown by dashed lines in FIG. 2.

In addition, a heartbeat volume variability can be determined or obtained for each measured value of the measurement data 5. The measurement data 5 can be adjusted based on the heartbeat volume variability to account for fluctuations in heartbeat volume.

In particular, the heartbeat volume variability can be used to correct a measured value inversely proportionally to the fluctuation. If a heartbeat has pumped unusually little/much volume and a heartbeat otherwise typically pumps more/less blood, this can be corrected upwardly/downwardly.

Subsequently, the cardiovascular performance 2 is provided by an output interface 8. The output interface 8 can include an interface for transferring the cardiovascular performance 2 or a display for outputting the cardiovascular performance.

Alternatively, the measurement data 5 can be used to provide a slope metric, such as the average slope of the measurement data 5 after blood flow is restored until a saturation value is reached. The time interval of the measurement data 5 can be selected as described above. In addition, a characteristic value can be determined. The characteristic value is, for example, an arithmetic metric of the measurement data 5 within the time interval or after a saturation value has been reached. The slope metric and the characteristic value can be used to model the increasing blood perfusion, which in turn is proportional to the cardiovascular performance. Thus, the slope metric and the characteristic value can be offset, e.g. multiplied, and used by a mapping, e.g. a lookup table/function, to determine the cardiovascular performance. The mapping is determined by comparable measured values of cardiovascular performance, preferably by magnetic resonance imaging. Preferably, the mapping is also configured to take into account the pulse rate of the patient.

FIG. 3 shows a plurality of perfusion amplitudes of the measurement data Y1 over time, which were determined at rest of the patient, i.e. at a resting pulse rate of the patient without a physical exertion. The measurement data Y1 were measured immediately after allowing blood flow to the distal periphery. A perfusion amplitude value was determined by normalizing the perfusion index measurement value measured with a sensor with a measurement value measured after 20 seconds. This results in a curve of the measurement data Y1 that rises steeply and reaches a saturation range after just a few seconds and fluctuates only slightly in this saturation range. The fit function Y1′ from FIG. 3 can be used to approximate the entire measurement or just the first 20 seconds of the measurement. The measurement data Y1 in FIG. 3 was fitted by a fourth-order polynomial fit function Y1′ so that a continuous function can be used to determine the cardiovascular performance.

However, the time interval for a fit function Y1′ in FIG. 3 can be adjusted so that perfusion amplitudes in the saturation range, which are subject to natural fluctuations in the measured values, are not overly taken into account. The time interval in FIG. 3 for determining the fit function Y1′ can be restricted approximately to the dashed-framed area in FIG. 3. This can be achieved by selecting the time interval such that four immediately consecutive perfusion amplitude measured values are below a predefined fraction of the subsequent perfusion amplitude value, for example at a predefined fraction of 95% of the subsequent perfusion amplitude value. The transient range in FIG. 3 therefore overlaps with the saturation range, i.e. a saturation value is assumed directly after a transient, in that the measurement data only fluctuates slightly around a saturation value. However, the measurement data, such as perfusion amplitude data, may increase again in the transient range or after the transient range, especially for large cardiovascular performances and/or if the measurement data was recorded during physical activity, until a saturation range is finally reached. The transient range can have one or more overshoots, as can be seen in FIG. 3 and FIG. 4B.

Based on such a fit function Y1′, the control unit can determine the cardiovascular performance.

FIG. 4A shows a plurality of perfusion amplitudes of the measurement data W1 over time, which were determined at rest of the patient. The measurement data W1 was also fitted with an exponential function1−e^{-Ct} W1′ as an example to determine the cardiovascular performance. The parameter t is the elapsed time after blood flow is restored, and C is a fit constant. A value of perfusion amplitude was determined by normalizing the perfusion index reading measured with a sensor with a reading taken after 20 seconds.

FIG. 4B shows a plurality of perfusion amplitudes of the measurement data W2 over time with overshoots determined at rest of the patient and approximated by a logarithmic fit function W2′. The measurement data W2 show that the perfusion amplitudes exhibit a “transient behavior”, i.e. fluctuate around the logarithmic fit function W2′ until a saturation value is finally reached. A value of the perfusion amplitude was determined by normalizing the perfusion index reading measured with a sensor with a reading taken after 30 seconds.

FIG. 5 shows a plurality of perfusion amplitudes of the measurement data X1 over time, determined when the patient was subjected to a physical exertion of 150 W. The perfusion amplitudes over time were approximated by a logarithmic fit function X1′. FIG. 5 also shows that a significantly longer time interval is necessary to reach a saturation range of the perfusion amplitude. For such a case, a different fit function a logarithmic fit function may provide a better approximation of the cardiovascular performance. Since cardiovascular performance is significantly higher during physical exertion, it takes more time for the perfusion amplitude to reach a saturation range. The perfusion amplitude was obtained by metric of a perfusion index reading after 35 seconds.

In a preferred embodiment, a series of measurements can be recorded for a patient both at rest and during physical exertion, in particular 150 W, so that the cardiovascular performance can be determined as a function of the corresponding pulse rate.

FIG. 6 shows a training data set, exemplary comprising five measurement series X1-X5 of perfusion amplitudes, for training the machine learning model 12 over time. The training data set also preferably comprises the pulse rate at which the respective measurement series X1-X5 was determined. The training data set may further comprise a metric for measuring the physical exertion at which the respective measurement series was recorded, in order to improve the determination of the cardiovascular performance, which depends on the pulse rate. The perfusion amplitudes of the measurement series in FIG. 6 show exemplary very different curves, as they were recorded at different levels of physical exertion and for different patients. The measurement series X1 and X2 were recorded at rest, i.e. without physical exertion, while X3-X5 were recorded during physical exertion. In order for the machine learning model to provide sufficiently good predictions of cardiovascular performance, many such series of measurements must be taken, the target values determined and the machine learning model trained.

These measurement series X1-X5 are assigned target values of cardiovascular performance, which were preferably determined by other known methods for determining cardiovascular performance. The target values of the cardiovascular performance can be determined, for example, by known methods such as thermodilution, the MRI method, the Fick principle, Doppler ultrasound, pulse contour analysis, VO₂max method, or bioimpedance measurement. Thus, the machine learning model 12 can be trained using these measurement series X1-X5 and target values to reliably determine cardiovascular performance. Several machine learning models can be trained and used, each of which was determined with specific target values of the cardiovascular performance of a known procedure, for example with MRI target values and ultrasound target values of the cardiovascular performance.

The training data set also has heartbeat volume variability values as measurement data, so that fluctuations in heartbeat volume, for example due to the patient's breathing, can be taken into account by the machine learning model when determining cardiovascular performance.

In order to avoid “overfitting”, the machine learning model 12 can also be validated on a validation dataset that also comprises a plurality of such validation measurement series and validation target values but was not used for training.

1. A computer-implemented method for determining a cardiovascular performance comprising the steps of:
receiving a plurality of measurement data through a communication interface from a sensor at a measurement section of a distal periphery of a patient, wherein the measurement data is indicative of perfusion of blood flow at the measurement section, and wherein the plurality of measurement data comprises at least two different measurement data detected at different times after blood flow to the distal periphery has been restored,
determining the cardiovascular performance on the basis of the plurality of measurement data, and
providing output data by an output interface comprising the cardiovascular performance.

2. The computer-implemented method according to claim 1, wherein the plurality of measurement data for determining the cardiovascular performance over a time interval are one of the following:
selected from a range of 1 s to 60 s,
selected in such a way that a deviation of directly consecutive measurement data is always greater than 5%,
selected such that a mean value of, 5 measurement data measured immediately following one another in time is greater than a predefined fraction of 75%, of a measurement data measured in immediate succession.

3. The computer-implemented method according to claim 1, wherein a distance between a location of the obstructed blood flow and the measurement section is at least 3 cm.

4. The computer-implemented method according to claim 1, wherein the plurality of measurement data comprises at least one of the following:
measurement data indicative of oxygen saturation in the blood,
volumetric measurement data indicative of perfusion amplitude at the measurement section data,
plethysmography measurement data which indicate the detection of a pulsation, and
pulse rates.

5. The computer-implemented method according to claim 1, wherein the method further comprises the following steps:
determining a slope metric using the plurality of measurement data measured chronologically in succession at different times, wherein the slope metric is indicative of the slope of the plurality of measurement data,
determining a characteristic value from the measurement data, wherein the characteristic value is indicative of a saturation value or a metric of the measurement data, and
determining the cardiovascular performance based on the slope metric and the characteristic value.

6. The computer-implemented method according to claim 1, wherein the cardiovascular performance is determined using the measurement data and at least one mapping logic, wherein the at least one mapping logic was determined using an ultrasound method, a thermodilution method, a magnetic resonance imaging method, the Fick principle, a pulse contour analysis, VO₂max method, and/or bioimpedance measurement for measuring the cardiovascular performance.

7. The computer-implemented method according to claim 1, wherein a perfusion amplitude is determined from the plurality of measurement data.

8. The computer-implemented method according to claim 7, wherein the method further comprises using a stroke volume variability metric to correct the measurement data.

9. The computer-implemented method according to claim 7, further comprising determining the cardiovascular performance using at least one of the following algorithms:
at least one perfusion amplitude fit function comprising a polynomial fit function, a logarithmic fit function, an exponential fit function, an exponential fit function with oscillatory damping, a logistic growth model fit function, a logistic growth model fit function with oscillatory damping, a power law saturation fit function, a Hill fit function, a differential equation fit function, a differential equation fit function of a second order,
providing a plurality of reference functions, including time-measured measurement data of a similar or a same pulse rate, determining a deviation of the plurality of reference functions with the plurality of received measurement data or correlation values of the measurement data, and selecting a reference function of the plurality of reference functions with a least deviation or a most similar correlation value to the received measurement data,
a perfusion amplitude averaged over a time interval of a measurement, which is offset against the pulse rate, and
a machine learning mode using the perfusion amplitude.

10. The computer-implemented method according to claim 1, wherein a time distance between the first pressurization command and the second pressurization command is at least 15 s, or
the time distance is selected such that a measured value or a mean value of 2 measurement data measured in immediate succession in time is smaller than a predefined fraction of a measured value or mean value of 2 measurement data measured in immediate succession in time, which were measured before the pressurization.

11. The computer-implemented method according to claim 1, wherein the plurality of measurement data of the sensor are detected at rest and/or under physical exertion of the patient.

12. The computer-implemented method according to claim 11, further comprising a first determination of a first cardiovascular performance based on measurement data recorded at rest and a second determination of a second cardiovascular performance based on measurement data recorded under physical exertion of the patient.

13. A computer program product comprising instructions which, when the program is executed by a computer, cause the computer to perform the computer-implemented method according to claim 1.

14. A non-transitory computer-readable medium comprising instructions which, when executed by a computer, cause the computer to perform the computer-implemented method according to claim 1.

15. A system configured to perform the computer-implemented method of claim 1, comprising a unit for performing the computer-implemented method.

16. The system according to claim 15, wherein the system further comprises at least one sensor, connectable to the unit, for the distal periphery of a patient and, wherein the at least one sensor is configured to do at least one of the following measure a plurality of measurement data indicative of oxygen saturation in the blood, and measure a plurality of volumetric measurement data.

17. The system according to claim 16, wherein the at least one sensor comprises at least one of:
a finger pulse oximeter,
a wrist pulse oximeter,
a wearable watch, with an integrated pulse oximeter sensor,
a stationary pulse oximeter device with removable sensors, and
a skin patch pulse oximeter.

18. The system according to claim 15, wherein the system further comprises a cuff and the cuff (9) is pressurizable by a pressurization command of the unit to stop and restore blood flow to the distal periphery of the patient.

19. A method for determining a cardiovascular performance from a plurality of measurement data comprising the steps of:
pressurization of a cuff over a systolic blood pressure of a patient to inhibit a blood flow to a distal periphery of the patient,
pressurizing the cuff to a sub systolic blood pressure to allow blood flow to the distal periphery,
measuring a plurality of measurement data from a sensor at a measurement section of the distal periphery of the patient, the measurement data being indicative of perfusion at the measurement section, the plurality of measurement data comprising at least two different measurement data detected at different times after blood flow to the distal periphery has been restored, and
determining the cardiovascular performance on the basis of the plurality of measurement data.

20. A computer-implemented method for training a machine learning model for determining a cardiovascular performance comprising:
receiving an input training dataset comprising measurement data and a target value of a cardiovascular performance, the measurement data having been detected by at least one sensor at a measurement section of a distal periphery of a patient after restoring a blood flow of the distal periphery, and
performing a training process of the machine learning model by predicting an output value, comparing the output value with the target value, adjusting model parameters by a loss function,
iteratively repeating the training process for further input training datasets until a termination criterion is reached, and
providing a trained machine learning model.

21. A training dataset for use in the method of training a machine learning model according to claim 20, comprising at least one input training dataset.

22. The training data set according to claim 21, wherein the target value of the cardiovascular performance has been determined by at least one of the following methods:
an ultrasound method,
a thermodilution method,
a magnetic resonance imaging method,
the Fick principle,
a pulse contour analysis,
VO₂max method, and
a bioimpedance measurement.

23. The computer-implemented method according to claim 1, wherein the determined cardiovascular performance is selected from one of: stroke volume and cardiac output.

24. The computer implemented method according to claim 1 further comprising at least one of:
sending a first pressurization command to pressurize a cuff over a systolic blood pressure of a patient and inhibit blood flow to a distal periphery of the patient, and
